(19) United States
(12) Patent Application Publication
(10) Pub. No.: US 2015/0057698 A1
(43) Pub. Date: Feb. 26, 2015

(54) DETACHABLE TREATMENT DEVICE DELIVERY SYSTEM WITH NARROWED DIAMETER REGION AT ATTACHMENT ZONE

(71) Applicant: Cook Medical Technologies LLC, Bloomington, IN (US)

(72) Inventors: Karol Lagodzki, Bloomington, IN (US); Jeremy Schaeffer, Bloomington, IN (US); Elizabeth Theobald, Bloomington, IN (US)

(73) Assignee: Cook Medical Technologies LLC, Bloomington, IN (US)

(21) Appl. No.: 14/287,396

(22) Filed: May 27, 2014

Related U.S. Application Data
(60) Provisional application No. 61/868,624, filed on Aug. 22, 2013.

(51) Int. Cl. A61B 17/12

(52) U.S. Cl. CPC A61B 17/12113; A61B 17/1214; A61B 2017/12095
USPC 606/200

(57) ABSTRACT

A detachable treatment device delivery system includes a delivery sheath defining a lumen, a treatment device having a proximal attachment segment, which includes an enlarged outer diameter region, configured for receipt within the delivery sheath, and a deployment wire configured for receipt within the delivery sheath. A delivery configuration is defined by an overlap of a distal segment of the deployment wire and the enlarged outer diameter region within an attachment zone of the delivery sheath. The deployment wire and the treatment device have a combined outer diameter at the overlap, and, in the delivery configuration, the overlap is proximally spaced from a narrowed diameter region of the attachment zone, which has a smaller diameter than the combined outer diameter. In a deployed configuration, the deployment wire is proximally spaced from the attachment zone, and the proximal attachment segment is permitted to advance through the distal opening.

### TECHNICAL FIELD

The present disclosure relates generally to a delivery system for detachable treatment devices, and more particularly to a delivery sheath including a narrowed diameter region of an attachment zone sized to restrict distal movement of an overlap of a deployment wire and an enlarged outer diameter region of a treatment device.

### BACKGROUND

Medical treatment devices, such as, for example, embolization coils, microcoils, and plugs, are used to restrict or block blood flow to arterio-venous malformations and other vascular lesions. For example, these treatment devices may be delivered into an aneurysm of a patient to prevent blood from entering the aneurysm. Embolization is typically a non-surgical, minimally invasive procedure that involves pushing or injecting the treatment devices, or, alternatively, detaching the treatment devices at the treatment site. Both delivery methods offer benefits and suffer drawbacks, particularly depending on the specifics of the procedure being performed.

According to one example, U.S. Pat. No. 8,333,796 to Tompkins et al. discloses embolic coil implant systems and methods for mechanically retaining and releasing coils. According to one embodiment, an urging means and aperture are disposed within a sidewall of an implant tool. A filament is disposed within a lumen of the implant tool and pushes a portion of a coil into the aperture, against the urging means, to releasably retain a proximal portion of the coil. When the filament is withdrawn, the urging means presses the retained portion of the coil away from the aperture and into the lumen of the implant tool. The proximal portion of the coil is then released through a distal opening of the implant tool. Although the embolic coil deployment system of the Tompkins et al. reference may be suitable for some applications, there is a continuing need for improved deployment systems, including those that offer control, reliability, simplicity, and the ability to navigate tortuous anatomy.

The present disclosure is directed toward one or more of the problems or issues set forth above.

### SUMMARY OF THE DISCLOSURE

In one aspect, a detachable treatment device delivery system includes a delivery sheath defining a lumen extending from a proximal opening to a distal opening. A treatment device has a proximal attachment segment, which includes an enlarged outer diameter region, configured for receipt within the delivery sheath. The detachable treatment device delivery system also includes a deployment wire configured for receipt within the delivery sheath. A delivery configuration is defined by an overlap of a distal segment of the deployment wire and the enlarged outer diameter region of the treatment device within an attachment zone defined by the delivery sheath. The deployment wire and the treatment device have a combined outer diameter at the overlap, and, in the delivery configuration, the overlap is proximally spaced from a narrowed diameter region of the attachment zone. A diameter of the narrowed diameter region is less than the combined outer diameter. In a deployed configuration, the deployment wire is proximally spaced from the attachment zone, and the proximal attachment segment is distally spaced from the attachment zone and the distal opening. A diameter of the enlarged outer diameter region is less than the diameter of the narrowed diameter region.

In another aspect, a method of deploying a treatment device using a detachable treatment device delivery system is provided. The detachable treatment device delivery system includes a delivery sheath defining a lumen extending from a proximal opening to a distal opening, and a deployment wire. The treatment device includes a proximal attachment segment having an enlarged outer diameter region. The method includes creating an overlap of a distal segment of the deployment wire with the enlarged outer diameter region of the treatment device within an attachment zone defined by the delivery sheath. The deployment wire and the treatment device have a combined outer diameter at the overlap. Distal movement of the treatment device is restricted using a narrowed diameter region of the attachment zone. The overlap is proximally spaced from the narrowed diameter region and a diameter of the narrowed diameter region is less than the combined outer diameter. The method also includes proximally retracting the deployment wire within the delivery sheath such that the deployment wire is proximally spaced from the attachment zone, and releasing the treatment device from the delivery sheath through the distal opening responsive to the proximally retracting step such that the proximal attachment segment of the treatment device is distally spaced from the attachment zone and the distal opening. A diameter of the enlarged outer diameter region is less than the diameter of the narrowed diameter region.

### BRIEF DESCRIPTION OF THE DRAWINGS

FIG. 1 is a partially sectioned side diagrammatic view of a detachable treatment device delivery system, shown in a delivery configuration, according to one embodiment of the present disclosure;

FIG. 2 is an end view facing a distal end of the detachable treatment device delivery system of FIG. 1;

FIG. 3 is a partially sectioned side diagrammatic view of the detachable treatment device delivery system of FIG. 1, shown in a deployed configuration;

FIG. 4 is a partially sectioned side diagrammatic view of a detachable treatment device delivery system, according to another embodiment of the present disclosure;

FIG. 5 is a partially sectioned side diagrammatic view of a detachable treatment device delivery system, according to another embodiment of the present disclosure; and

FIG. 6 is a partially sectioned side diagrammatic view of a detachable treatment device delivery system, according to another embodiment of the present disclosure.

### DETAILED DESCRIPTION

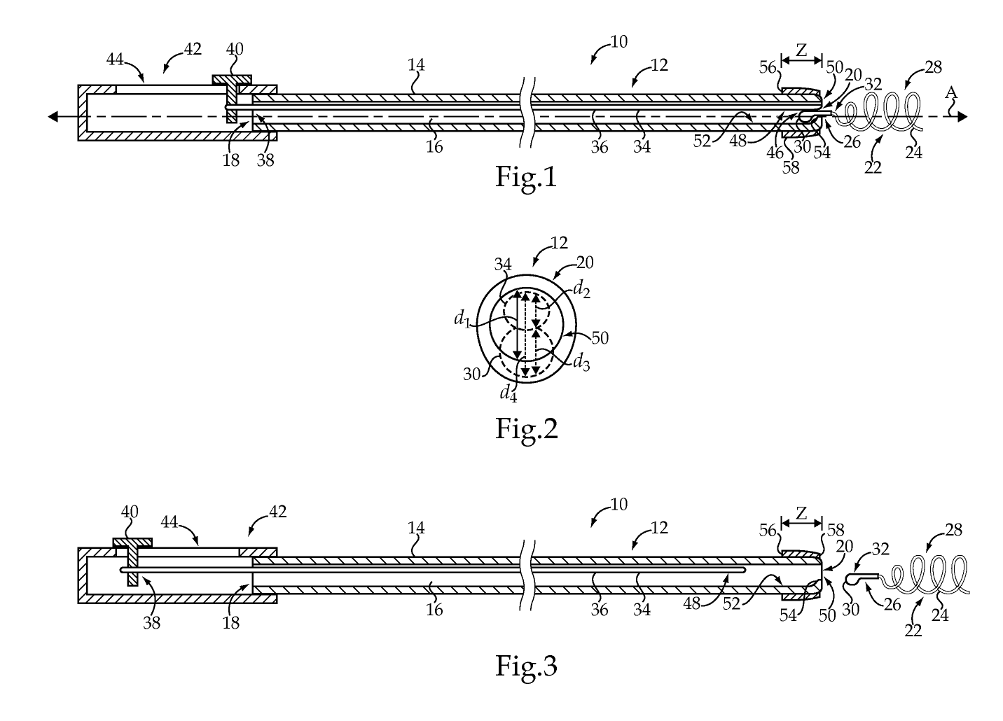

Referring to FIG. 1, there is shown a detachable treatment device delivery system 10 according to one embodiment of the present disclosure. Although not shown, the detachable treatment device delivery system 10 may be provided within a sterile, tear open package, as is known in the art. In performing an embolization procedure on a patient, some or all of the components of the detachable treatment device delivery system 10 may be used, depending upon the specifics of the procedure to be performed. As should be appreciated, however, the components shown in FIG. 1 might be separately packaged and/or the detachable treatment device delivery system 10 might also include components in addition to those shown, including components routinely used in percutaneous vascular procedures.

The detachable treatment device delivery system 10 includes a delivery sheath 12 having an elongate tubular body 14 defining a lumen 16 extending from a proximal opening 18 to a distal opening 20. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

The elongate tubular body 14 may be made from any common medical tube material, such as, for example, polytetrafluoroethylene (PTFE), high density polyethylene (HDPE), nylon, polyetheretherketone (PEEK), or any vinyl, plastic, rubber, silicone, or metal and may exhibit both stiffness, or firmness, and flexibility. Materials as well as dimensions may vary depending on the particular application. However, it may be desirable to dimension the delivery sheath 12 for receipt within a particular catheter or microcatheter. For example, the delivery sheath 12 may have an outer diameter, according to all potential configurations, that is less than 0.035 inch, or other selected catheter inner diameter. A length of the delivery sheath 12 may be between about 50 centimeters to about 225 centimeters, which may correspond to a delivery catheter with which the detachable treatment device delivery system 10 is to be used.

A treatment device 22, which may, for example, be an embolization coil 24, has a proximal attachment segment 26 configured and sized for receipt within the delivery sheath 12. According to a delivery configuration, as shown in FIG. 1, the proximal attachment segment 26 may be disposed within the lumen 16 of the delivery sheath 12, while a distal end 28 of the treatment device 22 is distally disposed relative to the distal opening 20 of the delivery sheath 12. As is known to those skilled in the art, all or a portion of the embolization coil 24 may have a preformed coiled shape. According to some embodiments, the preformed coiled shape may be straightened in the delivery configuration. Although the shapes and sizes of coils may vary greatly, it should be appreciated that the embolization coil 24 may have an outer diameter, according to the preformed coil shape, between about 0.005 and 0.05 inch and may have a length of between about 1.5 to 30 centimeters.

Although treatment device 22 is exemplified as an embolization coil, it should be appreciated that various other treatment devices, including plugs, may be used with the detachable treatment device delivery system 10 instead of the embolization coil 24. To accommodate use with the detachable treatment device delivery system 10, the treatment device 22 preferably includes the proximal attachment segment 26, which is configured for receipt within the delivery sheath 12. In particular, the proximal attachment segment 26 should be sized and shaped for receipt within the delivery sheath 12 and configured for detachable retention within the delivery sheath 12 as described herein. The proximal attachment segment 26 may be integral with the distal end 28, or the proximal attachment segment 26 and the distal end 28 may be separate components that are attached together using known attachment means.

According to the exemplary embodiment, the proximal attachment segment 26 includes an enlarged outer diameter region 30. For reasons that will become more apparent below, the enlarged outer diameter region 30 is enlarged relative to a distal portion 32 of the proximal attachment segment 26. That is, the enlarged outer diameter region 30 has a larger diameter and/or area than the distal portion 32 of the proximal attachment segment 26 that is received through the distal opening 20 of the delivery sheath 12 in the delivery configuration. The proximal attachment segment 26 may be made from similar materials as the distal end 28 of the treatment device 22 or may be made from different materials than the distal end 28.

The detachable treatment device delivery system 10 also includes a deployment wire 34 configured for receipt within the delivery sheath 12. The deployment wire 34 may have an elongate body 36 made from any of a variety of medical grade materials, including those identified with respect to the delivery sheath 12. As shown in the delivery configuration of FIG. 1, the deployment wire 34 may be disposed within the lumen 16 of the delivery sheath 12 and movable along a longitudinal axis A of the delivery sheath 12. In particular, for example, a proximal end 38 may be attached to, or configured to move with, a movable portion 40 of a handle 42. The handle 42 may be disposed over the proximal opening 18 of the delivery sheath 12 and may include an elongate slot 44 within which the movable portion 40 may be refracted. Although a simplified version of handle 42 is shown, it should be appreciated that a variety of handles, ranging in complexity, may be adapted for use with the detachable treatment device delivery system 10.

The delivery configuration of FIG. 1 is defined by an overlap, shown at 46, of a distal segment 48 of the deployment wire 34 and at least the enlarged outer diameter region 30 of the treatment device 22 along the longitudinal axis A. The overlap 46, which may include direct contact of the deployment wire 34 and the treatment device 22, is positioned within an attachment zone Z defined by the delivery sheath 12. The attachment zone Z may have a length corresponding to, or including, portions of the deployment wire 34 and the treatment device 22 that are overlapped. Also according to the delivery configuration, the overlap 46 is proximally spaced from a narrowed diameter region 50 of the attachment zone Z. According to one example, the narrowed diameter region 50 may be defined by a wall 52 that defines the lumen 16 of the delivery sheath 12. In particular, a projection or protrusion 54 of the wall 52 may define the narrowed diameter region 50 and, according to some embodiments, the protrusion 54 may be aligned with and/or shaped to engage the enlarged outer diameter region 30 of the treatment device 22.

The narrowed diameter region 50 may coincide with the distal opening 20 of the delivery sheath 12 and, as shown in FIG. 2, a diameter d₁ of the narrowed diameter region 50 may be the same as that of the distal opening 20. A diameter d₂ of the deployment wire 34 and a diameter d₃ of the treatment device 22, both taken at the overlap 46, are shown in broken lines. Thus, it can be seen in FIG. 2 that a combined outer diameter d₄, of the deployment wire 34 and the enlarged outer diameter region 30 of the treatment device 22 at the overlap 46, is greater than the narrowed diameter region 50 of the delivery sheath 12. According to the delivery configuration, the narrowed diameter region 50 restricts distal movement of the treatment device 22. In particular, with the deployment wire 34 and the treatment device 22 in an overlapped position and the overlap 46 being proximally spaced from the narrowed diameter region 50, the enlarged outer diameter region 30 is restricted from advancing distally through the distal opening 20.

According to some embodiments, a compressive band 56 may be positioned over the delivery sheath 12 at the attachment zone Z and/or the narrowed diameter region 50. The compressive band 56 may assist in maintaining the diameter d₁ of the narrowed diameter region 50 and/or, according to some embodiments, may define all or a portion of the narrowed diameter region 50. The compressive band 56 may be, or may include, a radiopaque marker 58 to facilitate fluoroscopic visualization of the attachment zone Z and/or the narrowed diameter region 50. Additional or alternative radiopaque markings may be provided on components of the detachable treatment device delivery system 10, including radiopaque markings on portions of the deployment wire 34 and/or treatment device 22.

The handle 42 may be manipulated to move the detachable treatment device delivery system 10 from the delivery configuration of FIG. 1 to a deployed configuration, as shown in FIG. 3. In particular, the movable portion 40 of the handle 42 may be proximally retracted to correspondingly retract the deployment wire 34. According to the deployment configuration, the deployment wire 34 is proximally spaced from the attachment zone Z, and the proximal attachment segment 26 of the treatment device 22 is distally spaced from the attachment zone Z and the distal opening 20. That is, once the deployment wire 34 is retracted such that the distal segment 48 of the deployment wire 34 and the proximal attachment segment 26 of the treatment device 22 are no longer overlapped, the enlarged outer diameter region 30 of the proximal attachment segment 26 of the treatment device 22 is permitted to advance through the distal opening 20. Accordingly, the diameter d₃ of the enlarged outer diameter region 30 is less than the diameter d₁ of the narrowed diameter region 50.

As stated above, one or more portions of the proximal attachment segment 26, which includes the enlarged outer diameter region 30, may be urged against a preformed coiled shape in a delivery configuration. Alternatively, portions of the proximal attachment segment 26 may have a preformed straightened shape that remains straightened after the treatment device 22 has been deployed. According to the deployed configuration, one or both of the distal end 28 and the proximal attachment segment 26 may conform to a preformed coiled shape, as is known to those skilled in the art.

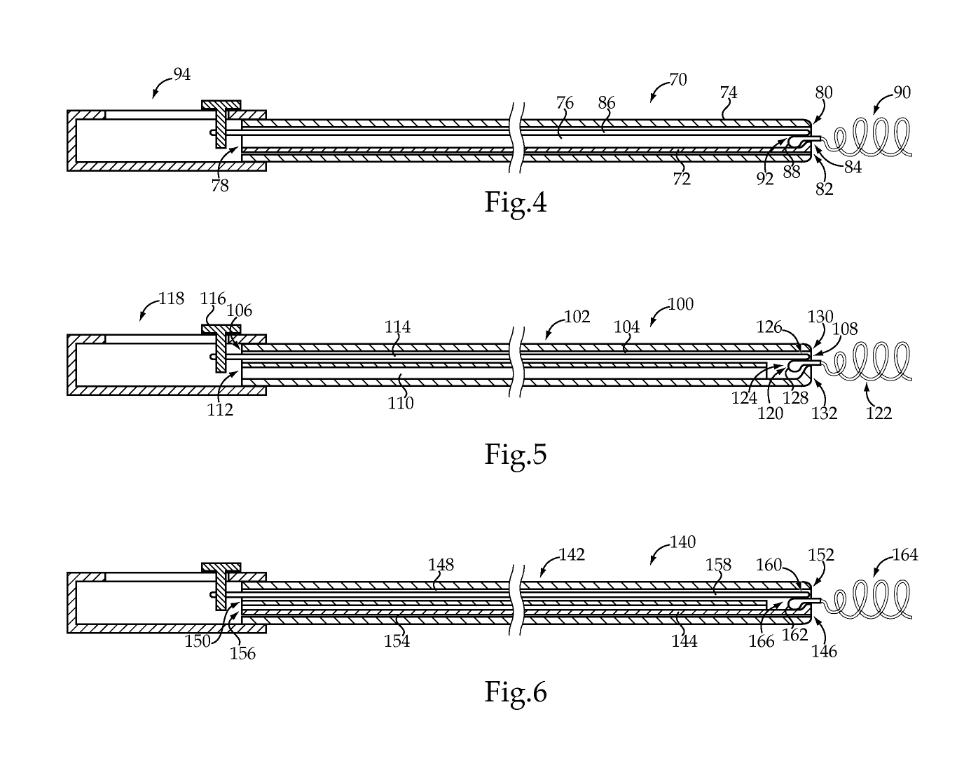

According to an alternative embodiment, as shown in FIG. 4, a detachable treatment device delivery system 70 according to the present disclosure may include an additional wire 72. In particular, the detachable treatment device delivery system 70 may include a delivery sheath 74 defining a lumen 76 extending from a proximal opening 78 to a distal opening 80. The additional wire 72 may be received within, and may be stationary with respect to, the delivery sheath 74, and a distal segment 82 of the additional wire 72 may define a narrowed diameter region 84 of the delivery sheath 74. A deployment wire 86 and an enlarged outer diameter region 88 of a treatment device 90 may be overlapped at an overlap 92. According to a delivery configuration, the overlap 92 is proximally spaced from the narrowed diameter region 84, such that the treatment device 90 is releasably engaged with the delivery sheath 74. A handle 94 may retract the deployment wire 86 to permit distal advancement of the enlarged outer diameter region 88 through the distal opening 80.

According to an alternative embodiment, as shown in FIG. 5, a detachable treatment device delivery system 100 according to the present disclosure may include a dual lumen sheath 102. That is, the delivery sheath 102 may define a first lumen 104 extending from a first proximal opening 106 to an open distal end 108, and a second lumen 110 extending from a second proximal opening 112 to the open distal end 108. The lumens 104 and 110 may be arranged in various configurations, including a side-by-side configuration or a coaxial configuration. A deployment wire 114 may be attached to a movable portion 116 of a proximal handle 118 and may be movable within the first lumen 104. A proximal attachment segment 120 of a treatment device 122 may be disposed through the open distal end 108 and within a common area 124 that is proximally spaced from the open distal end 108. In a delivery configuration, a distal segment 126 of the deployment wire 114 and an enlarged outer diameter region 128 of the treatment device 122 are overlapped at an overlap 130 and may be in direct contact within the common area 124 of the delivery sheath 102. To restrict distal movement of the treatment device 122 in the delivery configuration, the overlap 130 is proximally spaced from a narrowed diameter region 132. Proximal retraction of the deployment wire 114 permits release of the treatment device 122 relative to the delivery sheath 102.

Turning now to FIG. 6, another alternative embodiment is shown. In particular, a detachable treatment device delivery system 140 may include a dual lumen sheath 142 that is similar to the dual lumen sheath 102 of FIG. 5. However, the dual lumen sheath 142 of FIG. 6 includes an additional wire 144 that defines a narrowed diameter region 146. The sheath 142 defines a first lumen 148 extending from a first proximal opening 150 to an open distal end 152 and a second lumen 154 extending from a second proximal opening 156 to the open distal end 152. In the delivery configuration, a deployment wire 158 is received within the first lumen 148 and the additional wire 144 is received within the second lumen 154. The additional wire 144 may or may not be stationary with respect to the sheath 142 but, according to the delivery configuration, an overlap 160 of the deployment wire 158 and an enlarged outer diameter region 162 of a treatment device 164 occurs in a common area 166 of the sheath 142 that is proximally spaced from the narrowed diameter region 146.

### INDUSTRIAL APPLICABILITY

Referring generally to FIGS. 1-6 and more specifically to the embodiment of FIGS. 1-3, a method of deploying the treatment device 22 using the detachable treatment device delivery system 10 will be described. To load the detachable treatment device delivery system 10 and place the same in a delivery configuration, the proximal attachment segment 26 of the treatment device 22 may be inserted through the distal opening 20 of the delivery sheath 12, with the deployment wire 34 retracted relative to the attachment zone Z. Once the enlarged outer diameter region 30 is positioned distally relative to the narrowed diameter region 50, the deployment wire 34 may be positioned to define the overlap 46. According to some embodiments, the deployment wire 34 may be positioned partially within or may be aligned with the narrowed diameter region 50 to assist in maintaining the releasable engagement of the treatment device 22 with the delivery sheath 12.

Those skilled in the art should appreciate that the detachable treatment device delivery system 10 may be provided in the preloaded state described above and may also include a delivery cannula for maintaining a straightened configuration of the distal end 28 of the treatment device 22 during delivery. The delivery cannula may be configured to retract during advancement of the detachable treatment device delivery system 10 through a delivery catheter, which may be a microcatheter. The delivery catheter, according to one example, may then maintain a straightened configuration of the distal end 28 of the treatment device 22 as the detachable treatment device delivery system 10 is advanced, in the delivery configuration, to a treatment site within a patient, such as, for example, a vascular structure.

When the detachable treatment device delivery system 10 is properly positioned, the handle 42 may be manipulated to proximally retract the deployment wire 34 within the delivery sheath 12 such that the deployment wire 34 is proximally spaced from the attachment zone Z. As such, the deployment wire 34 no longer blocks or restricts passage of the enlarged outer diameter region 30 distally through the distal opening 20. The treatment device 22 may be then be completely released from the delivery sheath 12 through the distal opening 20 in response to the retraction of the deployment wire 34 such that the proximal attachment segment 26, including the enlarged outer diameter region 30, of the treatment device 22 is distally spaced from the attachment zone Z and the distal opening 20.

The distal segment 48 of the deployment wire 34, the proximal attachment segment 26 of the treatment device 22, including the enlarged outer diameter region 30, and the narrowed diameter region 50 are sized and configured to provide the releasable engagement of the treatment device 22 with the delivery sheath 12 that is described herein. In particular, when the enlarged outer diameter region 30 and the deployment wire 34 are overlapped, the enlarged outer diameter region 30 is prevented from advancing proximally through the narrowed diameter region 50. According to some embodiments, the combined outer diameter d₄ at the overlap 46 may substantially match a diameter defined by the inner wall 52 at a position proximal to the narrowed diameter region 50. For example, it may be desirable for the wall 52 to frictionally engage the overlap 46 and reduce movement of the deployment wire 34 and the treatment device 22 in the delivery configuration. Alternatively, or additionally, it may be desirable to include additional features that restrict longitudinal movement of the deployment wire 34 and the treatment device 22 during delivery.

The detachable treatment device delivery system 10 provides a low profile means for reliably and precisely detaching a treatment device 22 at a treatment site. For example, the delivery sheath 12 may have a continuous, low profile outer diameter that may effectively advance through a relatively small catheter, such as a microcatheter. The detachable treatment device delivery system 10 may be relatively flexible to navigate tortuous anatomy, while reliably maintaining the releasable attachment of the treatment device 22 during the navigation. Control of the treatment device 22 remains after the detachable treatment device delivery system 10 is proximally advanced relative to the delivery catheter and until the deployment wire 34 is retracted to release the treatment device 22.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A detachable treatment device delivery system, comprising:
a delivery sheath defining a lumen extending from a proximal opening to a distal opening;
a treatment device having a proximal attachment segment configured for receipt within the delivery sheath, wherein the proximal attachment segment includes an enlarged outer diameter region; and
a deployment wire configured for receipt within the delivery sheath;
a delivery configuration defined by an overlap of a distal segment of the deployment wire and the enlarged outer diameter region of the treatment device within an attachment zone defined by the delivery sheath, wherein the deployment wire and the treatment device have a combined outer diameter at the overlap, wherein, in the delivery configuration, the overlap is proximally spaced from a narrowed diameter region of the attachment zone, wherein a diameter of the narrowed diameter region is less than the combined outer diameter;
a deployed configuration in which the deployment wire is proximally spaced from the attachment zone, and the proximal attachment segment is distally spaced from the attachment zone and the distal opening, wherein a diameter of the enlarged outer diameter region is less than the diameter of the narrowed diameter region.

2. The detachable treatment device delivery system of claim 1, wherein a wall defining the lumen of the delivery sheath defines the narrowed diameter region.

3. The detachable treatment device delivery system of claim 2, further including a compressive band positioned over the delivery sheath at the narrowed diameter region.

4. The detachable treatment device delivery system of claim 3, wherein the compressive band includes a radiopaque marker.

5. The detachable treatment device delivery system of claim 2, wherein:
the delivery sheath defines a single lumen; and
the distal segment of the deployment wire and the enlarged outer diameter region of the treatment device are in direct contact within the single lumen in the delivery configuration.

6. The detachable treatment device delivery system of claim 2, wherein:
the delivery sheath defines a first lumen extending from a first proximal opening to an open distal end and a second lumen extending from a second proximal opening to the open distal end; and
in the delivery configuration, the distal segment of the deployment wire and the enlarged outer diameter region of the treatment device are in direct contact within a common area of the delivery sheath that is proximally spaced from the open distal end.

7. The detachable treatment device delivery system of claim 1, further including an additional wire received within the delivery sheath, wherein a distal segment of the additional wire defines the narrowed diameter region.

8. The detachable treatment device delivery system of claim 7, wherein the delivery sheath defines a single lumen, and the distal segment of the deployment wire and the enlarged outer diameter region of the treatment device are in direct contact within the single lumen in the delivery configuration.

9. The detachable treatment device delivery system of claim 8, wherein the additional wire is stationary with respect to the delivery sheath.

10. The detachable treatment device delivery system of claim 7, wherein:
the delivery sheath defines a first lumen extending from a first proximal opening to an open distal end and a second lumen extending from a second proximal opening to the open distal end; and
in the delivery configuration, the deployment wire is received within the first lumen and the additional wire is received within the second lumen, and the distal segment of the deployment wire and the enlarged outer diameter region of the treatment device are in direct contact within a common area of the delivery sheath that is proximally spaced from the open distal end.

11. The detachable treatment device delivery system of claim 10, wherein the additional wire is stationary with respect to the delivery sheath.

12. The detachable treatment device delivery system of claim 1, further including a radiopaque marker attached to the delivery sheath at the attachment zone.

13. The detachable treatment device delivery system of claim 1, wherein, in the deployed configuration, at least a distal end of the treatment device conforms to a preformed coiled shape.

14. A method of deploying a treatment device using a detachable treatment device delivery system, the detachable treatment device delivery system including a delivery sheath defining a lumen extending from a proximal opening to a distal opening, and a deployment wire, wherein the treatment device includes a proximal attachment segment having an enlarged outer diameter region, the method comprising steps of:
creating an overlap of a distal segment of the deployment wire with the enlarged outer diameter region of the treatment device within an attachment zone defined by the delivery sheath, wherein the deployment wire and the treatment device have a combined outer diameter at the overlap;
restricting distal movement of the treatment device using a narrowed diameter region of the attachment zone, wherein the overlap is proximally spaced from the narrowed diameter region and a diameter of the narrowed diameter region is less than the combined outer diameter;
proximally retracting the deployment wire within the delivery sheath such that the deployment wire is proximally spaced from the attachment zone; and
releasing the treatment device from the delivery sheath through the distal opening responsive to the proximally retracting step such that the proximal attachment segment of the treatment device is distally spaced from the attachment zone and the distal opening, wherein a diameter of the enlarged outer diameter region is less than the diameter of the narrowed diameter region.

15. The method of claim 14, wherein the step of restricting distal movement of the treatment device includes restricting distal movement of the deployment wire and the treatment device at the overlap using a lumen wall of the delivery sheath that defines the narrowed diameter region.

16. The method of claim 14, wherein the step of restricting distal movement of the treatment device includes restricting distal movement of the deployment wire and the treatment device at the overlap using a distal segment of an additional wire received within the delivery sheath that defines the narrowed diameter region.

17. The method of claim 14, wherein the step of releasing the treatment device from the delivery sheath includes conforming at least a distal end of the treatment device to a preformed coiled shape.

18. The method of claim 14, further including manipulating a handle disposed over the proximal opening of the delivery sheath to proximally retract the deployment wire.
